(19) United States
(12) Patent Application Publication
(10) Pub. No.: US 2006/0282013 A1
(43) Pub. Date: Dec. 14, 2006

(54) Analyte-taking device

(75) Inventors: Myriam Kauffmann, Paris (FR); Pascal Simon, Vitry Sur Seine (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/505,975

(22) Filed: Aug. 18, 2006

Related U.S. Application Data
(62) Division of application No. 10/674,463, filed on Oct. 1, 2003.
(60) Provisional application No. 60/428,702, filed on Nov. 25, 2002.

(30) Foreign Application Priority Data
Oct. 1, 2002 (FR) 02 12156

(51) Int. Cl. A61B 10/00

(52) U.S. Cl. 600/572

(57) ABSTRACT

An analyte-taking device includes a tube provided at one end with an analyte-taking element; a plug inside the tube; and at least one liquid contained in an inside space of the tube separated from the analyte-taking element by the plug. The plug can be arranged, in use, to be expelled together with the liquid when the liquid leaves the inside space of the tube. The liquid can be an analyte-taking liquid suitable for facilitating the taking of at least one analyte by the analyte-taking element. The liquid can also be a reagent suitable for producing an observable reaction in the presence of an analyte picked up by the analyte-taking element.

### CROSS-REFERENCE TO RELATED APPLICATIONS

This application is a divisional of application Ser. No. 10/674,463, filed Oct. 1, 2003, which claims priority to French Application No. 02 12156, filed Oct. 1, 2002 and U.S. Provisional Application No. 60/428,702, filed Nov. 25, 2002, the entire contents of all of which are hereby incorporated by reference.

### FIELD OF THE INVENTION

The present invention relates to analyte-taking devices, and more particularly but not exclusively to devices for taking an analyte from the surface of a tissue, e.g. a skin. The analyte can be, for example, an ion, an organic compound, or biological material. The invention can be used, for example, to facilitate analyte-taking for the purpose of establishing a diagnosis or an evaluation of the state of an individual, and in particular of the state of that individual's skin.

### BACKGROUND OF THE INVENTION

Discussion of Background

U.S. Pat. No. 3,958,571 discloses an applicator with a tube containing a liquid and having an applicator element at one end of the tube. Such an applicator is designed to apply medication such as a solution of iodine.

### SUMMARY OF THE INVENTION

There exists a need for an analyte-taking tool which is easy to transport and capable of being made at low cost so as to be suitable for single use.

In one embodiment, the invention provides an analyte-taking device including a container provided at one end with an analyte-taking element. The container can be a tube for example. A plug can be provided inside the tube. A liquid inside the tube can be separated from the analyte-taking element at least by the plug. The plug can be arranged, in use, to be expelled together with the liquid towards the analyte-taking element.

The liquid can be, for example, an analyte-taking liquid suitable for facilitating the taking of at least one analyte by the analyte-taking element. For example, the analyte-taking liquid can impregnate the analyte-taking element so as to make it suitable for use on a tissue of an individual for taking an analyte present at the surface of the tissue and enabling the analyte to be analyzed subsequently. The analyte-taking liquid can also be a reagent suitable for producing an observable reaction in the presence of a determined analyte picked up by the analyte-taking element.

The invention can provide a relatively simple and inexpensive arrangement for taking an analyte from the skin, for example, in order to perform a diagnosis or an evaluation. In addition, the invention can easily be used to ensure that the quantity of analyte-taking liquid or of reagent contained in the analyte-taking device is kept down to a sufficient minimum quantity by suitably selecting the dimensions of the tube.

The term “analyte” is used to designate a compound that is present at the surface of the skin, the mucous membranes, or the hair, or present in a body fluid such as urine, tears, saliva, or sweat, and that is capable of reacting with a specific reagent in order to be detected or quantified or in order to measure its activity. The analyte can be present in a cutaneous secretion such as sebum. The analyte can include microorganisms and/or substances associated therewith, whether dead or alive, and in particular it can include dead skin cells.

The analyte can also include lipids or enzymes present, for example, on the skin or on the mucous membranes. By way of example, the analyte can include ions, in particular carbonate ions, bicarbonate ions, calcium ions, chlorine ions, potassium ions, and metal cations such as Cu²⁺, Zn⁺, Fe²⁺, or Fe³⁺, for example. The analyte can also include organic compounds, in particular free amino acids, peptides, proteins, or hormones. The analyte can also include pollution residues, for example residues of arsenic, lead, mercury, iodine, or radioactive cesium.

The term “analyte-taking liquid” is used to designate any liquid enabling the analyte to be picked up or sampled, for example by dissolving it, fixing it, or transforming it. The analyte-taking liquid can include but is not be limited to: water; an aqueous solution; a water-alcohol solution; an oil solution; an oil; an organic solvent; chloroform; ethyl acetate; alcohols; chlorine-containing solvents; acetone; short esters; mixtures of water and methanol; and mixtures of chloroform and ethanol. It can also include an elutant and/or a fixer.

The term “reagent” is used to designate any compound, optionally in solution, that is suitable for reacting with a determined analyte in order to reveal its presence, or to quantify its concentration or its activity. By way of example, a reagent may change color or become bleached when coming into contact with a specific analyte. The reagent can be in the form of a powder, in the form of a gel, a hydrogel, a paste, or a liquid, amongst others.

When the device includes a reagent in the tube, the analyte-taking device can be pre-impregnated with an analyte-taking liquid, where appropriate.

The analyte-taking element can be porous, e.g. fibrous, e.g. in order to make it easy to impregnate with the analyte-taking liquid or the reagent.

By way of example, the analyte-taking element can be selected from: a cotton bud; a foam bud; a flocked bud; a felt tip; or a tip made of ceramic or sintered material; this list not being limiting.

The analyte-taking element can have adhesive or abrasive properties, e.g. for the purpose of taking a sample from the stratum corneum epidermis and/or elements present at the surface of the skin, a mucous membrane, the finger- or toe-nails, or the hair.

As mentioned above, the plug can include a liquid and/or a powder. When the plug includes a liquid, it can be selected from: mineral oils; fluorine-containing substances; and silicones, this list not being limiting.

By way of example, amongst the powders suitable for the plug, powders including organic or inorganic particles can be used. The particles can be solid or hollow, for example powders of microspheres of copolymers such as Expancel® (Nobel Industrie), of Nylon® (in particular Orgasol®), of waxes, of silicas, or of silicones, this list not being limiting.

The inside space of the tube can be defined at its end remote from the liquid or powder plug by a portion which can be broken off, removed, perforated, or deformed.

The analyte-taking device can be arranged in such a manner that after the break-off end has been broken off, the user can measure out the quantity of liquid that flows out by handling the tube as a pipette, while closing the top end of the tube with an index finger. The tube can optionally be sloping to a greater or lesser extent, where appropriate.

In an embodiment of the invention, the tube can be reclosed after only a fraction of the liquid contained inside it has moved out. Such reclosing can be performed, for example, using the break-off end. This end can be configured, for example, in such a manner as to be capable of constituting a closure plug, the break-off end including a spike suitable for engaging in the tube or on the tube in order to close it.

Where appropriate, the device can include a retaining element for retaining the break-off portion on the analyte-taking device after it has been broken off.

The volume of liquid contained in the tube can lie, for example, in the range from 0.01 milliliters (ml) to 5 ml, or in the range from 0.05 ml to 5 ml, or preferably in the range from 0.05 ml to 1 ml. The volume of liquid can be suitable for a single use of the analyte-taking device.

The tube of the analyte-taking device can be made of a transparent material, in particular of a transparent plastics material, so as to enable the user to observe the level of the liquid inside the tube or to observe its color, for example. The tube can include a multilayer structure, having at least one layer that forms a barrier against air, e.g. a layer of varnish impermeable to air, or to a solvent, or to ultraviolet (UV) radiation.

In another embodiment, the invention also provides a kit for sampling and analyzing an analyte. The kit can include at least one analyte-taking device. The device can include a tube with a plug inside the tube. The plug can be of liquid or powder.

The device can further include at least one analyte-taking liquid inside the tube within a space defined at a first end by the plug. An analyte-taking element is provided at one end of the tube, the analyte-taking element being separated from the analyte-taking liquid at least by the plug. The analyte-taking liquid can be suitable for facilitating the taking or sampling of at least one analyte.

The liquid or powder plug can be suitable for being expelled together with the analyte-taking liquid towards the analyte-taking element. The analyte-taking device can further include a reagent suitable, e.g. within the analyte-taking element, for producing an observable reaction in the presence of the analyte picked up by the analyte-taking element.

The kit can include various types of packaging. For example, the kit can include a box including at least one compartment in which at least one analyte-taking device is housed. In a variant, the kit can include at least one packaging bag containing at least one analyte-taking device. By way of example, the reagent can be present on strips contained in the box.

In another embodiment, the invention provides a method of detecting the presence and/or the concentration of at least one analyte, for example at the surface of tissue, such as the skin of an individual. The method can include a step of providing an analyte-taking device, which includes a tube, a plug inside the tube, an analyte-taking liquid inside the tube, and an analyte-taking element at one end of the tube, separated from the analyte-taking liquid by the plug. The method can further include a step of opening the tube so as to allow the analyte-taking liquid to leave the tube. The plug can be suitable for being expelled together with the analyte-taking liquid. A step of taking at least one analyte can be performed with the analyte-taking element so that the analyte can be placed into contact with a reagent suitable for producing an observable reaction in the presence of the analyte or in the presence of a determined concentration of the analyte.

The invention also provides a method of detecting the presence and/or the concentration of at least one analyte, for example an analyte present at the surface of the skin of an individual. The method can include a step of providing an analyte-taking device including a tube having a break-off portion at one end and an analyte-taking element at its other end and a plug of liquid or powder inside the tube. At least one reagent is provided inside the tube within a space defined at a first end by the plug. The tube can be broken so as to allow the reagent to impregnate the analyte-taking element. The plug can be suitable for being expelled together with the reagent towards the analyte-taking element. The reagent can reveal the presence of a determined analyte, optionally at a concentration that is greater than or less than a given threshold.

The invention also provides, independently or in combination with the above, a method of application of at least a substance contained in an inside space of the tube. The method can include heating the tube with a heat source before application of the substance. The substance can include, for example, at least a thermoreversible thickener so that the substance is reduced into a fluid state when the heat increases. The heat source can be the human heat or a heat source outside the human body, for example a source of hot water. Reducing the substance into a fluid state before application can improve the preservation of the substance during stocking in the tube, in particular by limiting the evaporation of the substance. Reducing the substance into a fluid state may also facilitate the passage of the substance through an application element, in particular an application element made of a porous material, for example cotton. The tube can, but need not, include a plug of one of a liquid and a powder disposed adjacent to the substance. This liquid or powder plug can be arranged to be expelled together with the substance when the substance leaves the inside space of the tube, in use.

### BRIEF DESCRIPTION OF THE DRAWINGS

Other characteristics and advantages of the invention will become apparent from the following detailed description, particularly when considered in conjunction with the drawings in which:

FIG. 1 is a diagram showing a kit for taking and analyzing an analyte;

FIGS. 2 to 4 show an analyte-taking device in isolation;

FIGS. 5 to 7 show various ways in which the analyte-taking device can be used;

FIG. 8 shows a portion of another example of a kit for taking and analyzing an analyte;

FIG. 9 shows another example of an analyte-taking device;

FIGS. 10 to 13 show variant embodiments of the analyte-taking element;

FIGS. 14 to 16 show various embodiments of the break-off portion of the analyte-taking device;

FIGS. 17 and 18 are diagrams showing analyte-taking devices that include two-phase or multi-phase reagents;

FIG. 19 is a diagrammatic and fragmentary view showing another example of an analyte-taking device;

FIG. 20 is a diagrammatic view of a receptacle suitable for receiving one or more analyte-taking devices; and

FIG. 21 is a diagrammatic view of a support for an analyte-taking device.

### DETAILED DESCRIPTION OF THE PREFERRED EMBODIMENTS

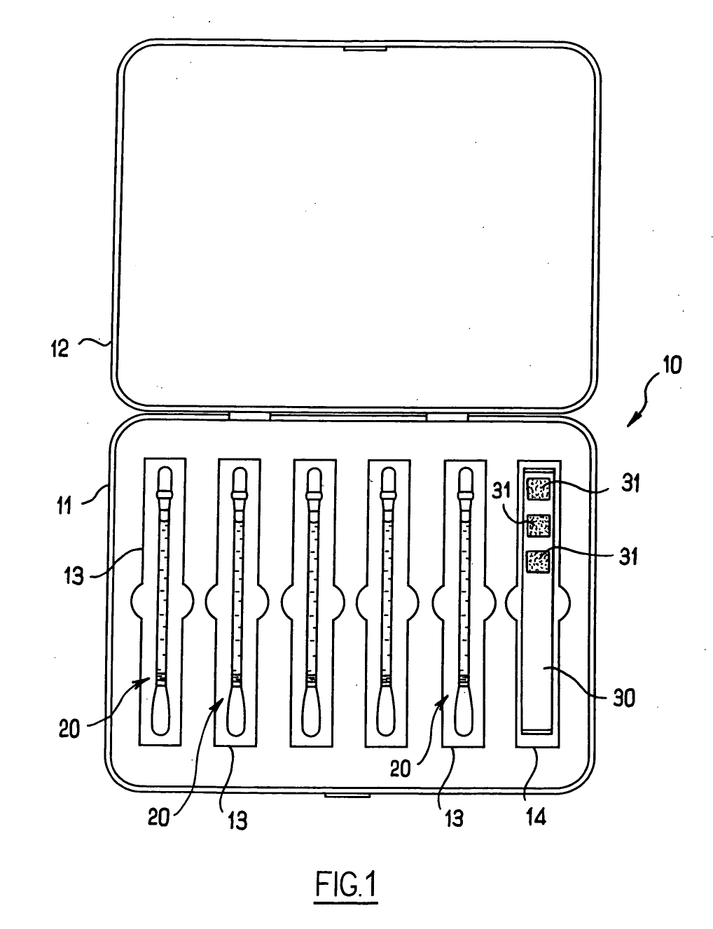

FIG. 1 is a diagram showing a kit for taking and analyzing an analyte including a box 10 having a base portion 11 and a lid 12 hinged to the base portion 11. The base portion 11 includes a plurality of compartments 13 housing analyte-taking devices 20. The base portion 11 further includes a compartment 14 housing a plurality of strips 30 each with one or more zones 31 impregnated with at least one reagent suitable for reacting with an analyte that can be taken with an analyte-taking device 20.

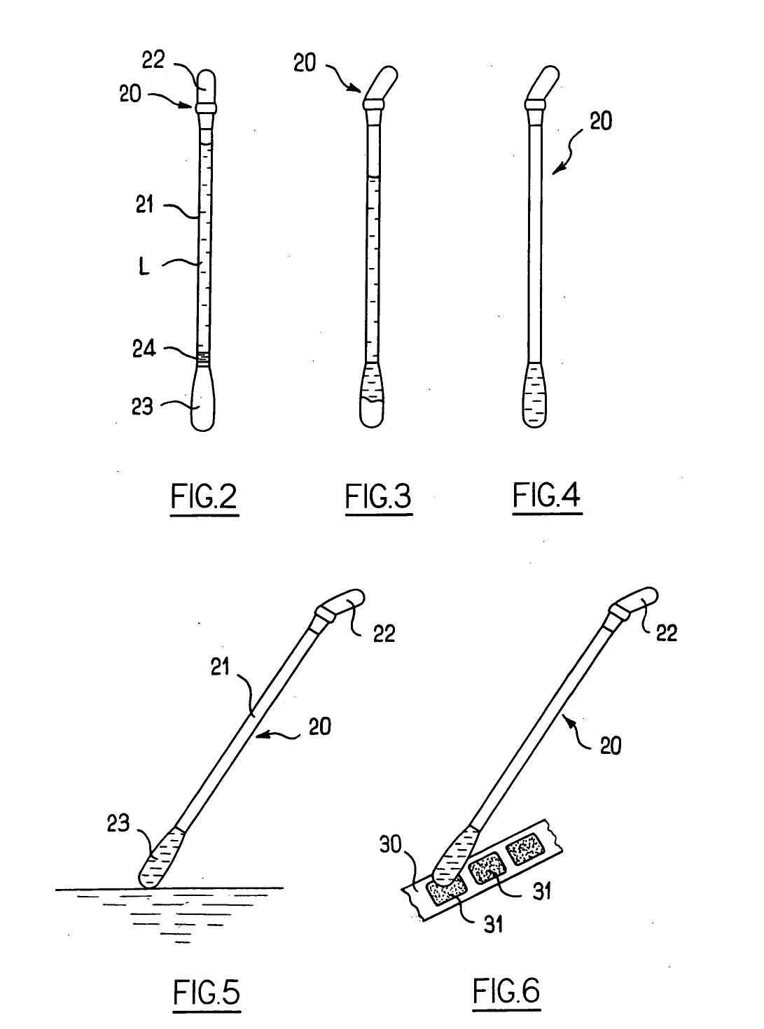

FIGS. 2 to 4 show an analyte-taking device 20 in isolation. By way of example, this device may be a device similar to the device described in U.S. Pat. No. 5,702,035, the entire content of which is hereby incorporated by reference. Applicators of that type are sold by the California supplier Swabplus Inc.

Each device 20 includes a container, for example a tube 21 containing an analyte-taking liquid L. For example, the tube can be made, in the assembly shown, by extruding a plastics material, and is provided at a closed end with a break-off portion 22. In the example described, this portion is covered by a cotton bud. The tube 21 is open at its other end remote from the break-off portion 22, being provided at this end with an analyte-taking element 23. In the example shown, the analyte-taking element 23 includes a cotton bud in the manner of a cotton applicator.

The analyte-taking liquid L is contained in an inside space of the tube 21 situated between the break-off portion 22 and a plug 24 present in the tube 21 beside its open end. The plug 24 can be a liquid or a powder.

The volume of analyte-taking liquid L can be suitable for a single use of the analyte-taking device, being determined as a function of the nature of the liquid and of the analyte to be taken. This volume can be in the range, for example, from 0.01 ml to 5 ml, and preferably in the range from 0.05 ml to 1 ml. The outside diameter of the tube 21 can be less than 6 millimeters (mm), for example, or even less than about 3 mm. The inside diameter of the tube 21 can lie in the range from about 0.5 mm to about 3 mm, for example.

When the plug 24 is formed by a liquid, the liquid of the liquid plug 24 can be any inert liquid compatible with packaging the liquid present in the tube 21. For example, the liquid can be a liquid that does not react with the analyte-taking liquid, that is capable of being expelled easily from the tube 21 at the time of use, and that is also physiologically acceptable. The liquid plug 24 serves in particular to isolate the analyte-taking liquid L from air, preventing it from evaporating and preventing external contaminants from penetrating. The liquid of the liquid plug 24 can be, for example, a mineral oil or a fluorine-containing substance, amongst other possibilities. In the example described, the liquid plug 24 is made of silicone. The quantity of liquid forming the liquid plug 24 is small compared with the quantity of analyte-taking liquid L.

When the break-off portion 22 is broken off, air can penetrate into the tube 21 through its end remote from its open end, and the analyte-taking liquid L can flow under gravity inside the tube 21, thereby reaching the analyte-taking element 23, as shown in FIGS. 3 and 4, so as to be put into contact with tissue of an individual from which an analyte is to be taken, for example, in contact with the skin.

In the example described, the optional cotton bud covering the break-off portion 22 serves to hold it to the remainder of the tube 21, even after it has been broken off.

In order to use the analyte-taking device 20, the break-off portion of the tube can be broken off, as shown in FIG. 5 so that the analyte-taking liquid L flows into the analyte-taking element 23.

This element is then brought into contact with the skin, for example, which may be lightly massaged so that the analyte that is to be revealed and that is present at the surface of the skin becomes deposited on the analyte-taking element or diffuses into the analyte-taking liquid L.

Once the analyte has been taken, the analyte-taking element can be brought into contact with a reagent-impregnated zone 31, as shown in FIG. 6. By way of example, the reagent may be configured so as to produce a colored reaction in the presence of a determined analyte as picked up by the analyte-taking element 23.

The analyte-taking liquid L can be water, for example, and in particular demineralized water, an aqueous solution, a water-alcohol solution, an alcohol solution, an oil solution, an oil, an organic solvent, being selected as a function of the analyte that is to be picked up, this list not being limiting.

The analyte whose presence on the surface of the skin is to be revealed can be, for example, an ion, in particular a carbonate, bicarbonate, calcium, or chlorine ion.

A strip 30 may include a plurality of zones 31, for example, each including reagents that react with different concentrations of a given analyte, so as to be capable of performing a quantitative measurement of the concentration of the analyte at the surface of the skin.

The liquid contained in the analyte-taking device may be an analyte-taking liquid as described with reference to FIGS. 1 to 6.

Alternatively, in the inside space situated between the plug 24 and the break-off portion 22, the tube 21 may contain a reagent in solution that is suitable for producing a reaction, and in particular a colored reaction, within the analyte-taking element 23 when in the presence of a determined analyte picked up by the analyte-taking element.

Under such circumstances, and as shown in FIG. 7, the analyte-taking element 23 can initially be impregnated with an analyte-taking liquid that is contained in a receptacle, e.g. it may be impregnated with demineralized water, and then the device 20 can be brought into contact with the skin in order to take any analyte that might be present thereon and that is suitable subsequently for reacting with the reagent contained in the tube 21, once the break-off portion of the device has been broken off. In another embodiment of the present invention, however, the analyte can also be taken from the surface of the skin without previously impregnating the analyte-taking element with a liquid. This embodiment can be useful, for example, when the analyte is contained in sebum or in a body fluid for example.

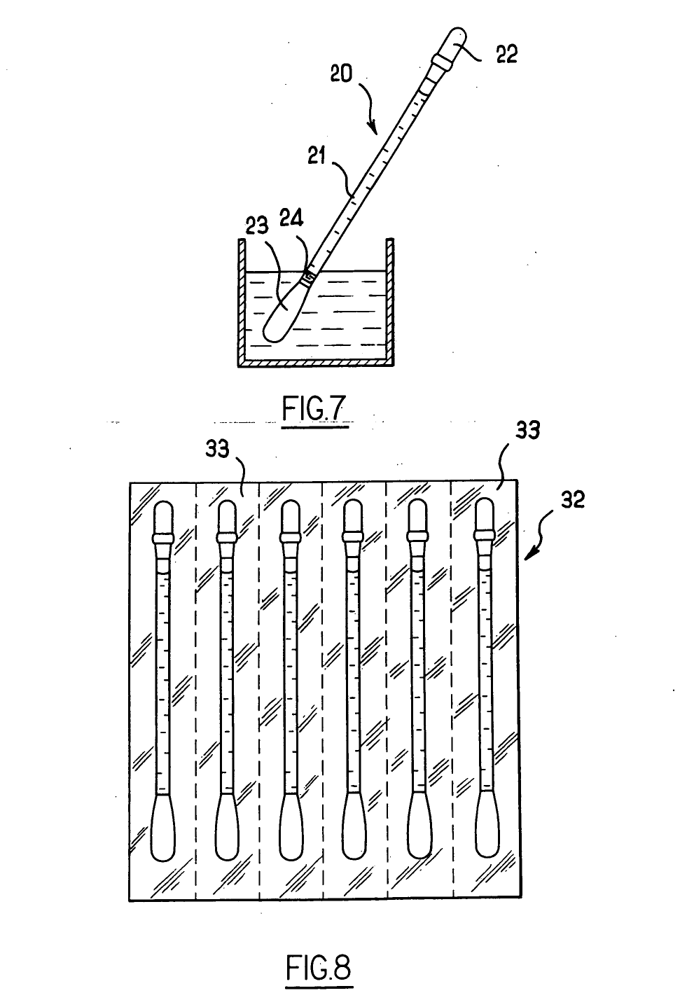

Analyte-taking devices can be packaged in a box as shown in FIG. 1, or they can be packaged in some other way without going beyond the scope of the present invention. For example, they can be packaged in the form of a string 32 of bags 33, as shown in FIG. 8.

The analyte-taking element can alternatively be pre-impregnated with an analyte-taking liquid as shown in FIG. 9. In which case, the liquid contained inside the tube 21 can contain a reagent suitable for revealing at least one analyte that might be present on the surface of the tissue of an individual. The analyte-taking device 20 of FIG. 9 can then be packaged in a hermetically sealed packaging bag 35 as shown in the figure.

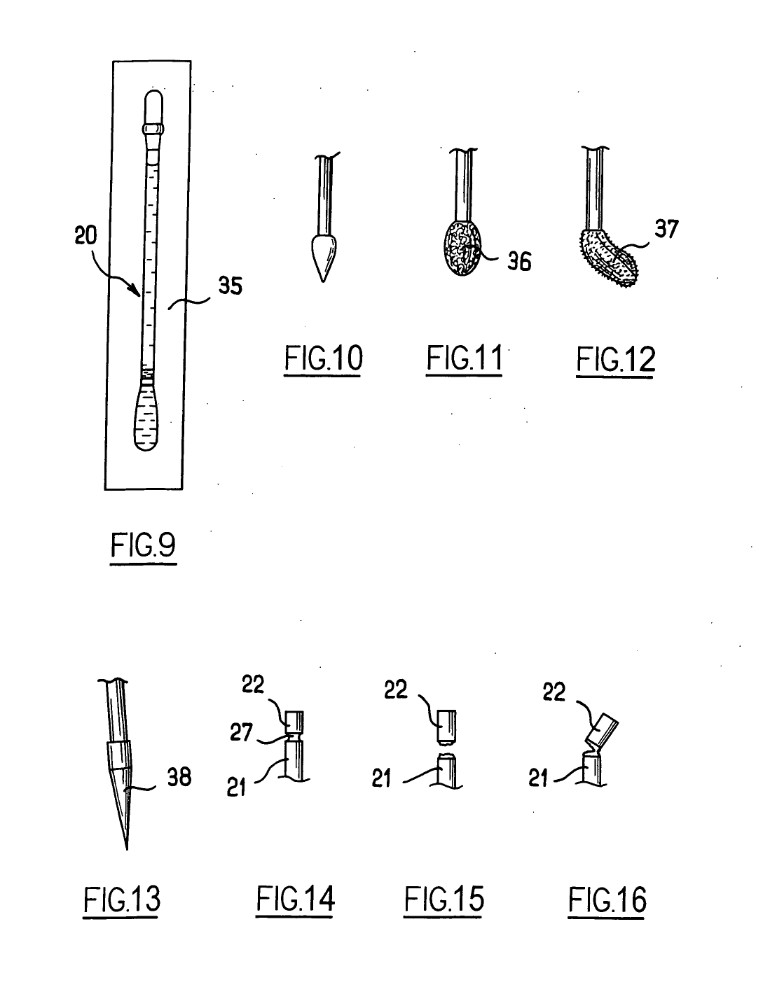

The analyte-taking element can be provided in a variety of shapes, and in particular it can have an end that is rounded or tapering. For example, it can be in the form of a pennon as shown in FIG. 10. By way of example, the analyte-taking element can be made of any porous material, e.g. fibrous material, and it may optionally be elastically compressible. By way of example, and as shown in FIG. 11, the analyte-taking element can be in the form of a foam bud 36.

The analyte-taking element can include flocking 37 on its surface, as shown in FIG. 12. This figure also shows that the analyte-taking element can be curved in shape, with a portion extending along a longitudinal axis that does not coincide with the axis of the tube 21. By way of example, the analyte-taking element can also be in the form of a bud 38 that is of tapering shape as shown in FIG. 13. Such a bud can be made of a porous material, or, in a variant, of a material that is not porous but that includes at least one internal channel or groove that enables the liquid contained in the tube 21 to flow towards the distal end.

The analyte-taking element can be made in such a manner as to be capable of exerting abrasive action, e.g. on the skin, in order to take cells. The analyte-taking element can be made of ceramic or of sintered material, for example. It is also possible for the analyte-taking element to be made by giving the tube a chamfered-shape, for example, thus enabling analytes to be taken by scratching. The analyte-taking element can also present adhesive properties.

FIGS. 14 to 16 show other embodiments of the break-off portion of the applicator. FIG. 14 shows that the break-off portion can be connected to the remainder of the tube via a preferred breakage zone 27, e.g. implemented in the form of a thinning in the wall of the tube or a notch at this level.

The break-off portion can be made in various other manners without going beyond the scope of the present invention. In particular, as shown in FIG. 15, the applicator can be configured in such a manner that the break-off portion 22 is capable of being completely separated from the tube 21 after manually applying a breaking movement by holding the tube 21 in one hand and the break-off portion 22 between two fingers of the other hand. The applicator can alternatively be configured in such a manner that, after use, the break-off portion 22 remains connected to the tube 21 via a bridge of material, as shown in FIG. 16. The end of the tube can be made in other ways, for example in one of the ways shown in FIGS. 3 to 8 of U.S. Pat. No. 3,958,571.

When the break-off end is capable of being completely detached from the tube, this feature can ease the use of the analyte-taking device as though it were a pipette, for example, with the user being capable of closing the top end of the analyte-taking element with a finger in order to dispense the liquid into the analyte-taking element in controlled manner, e.g. drop by drop.

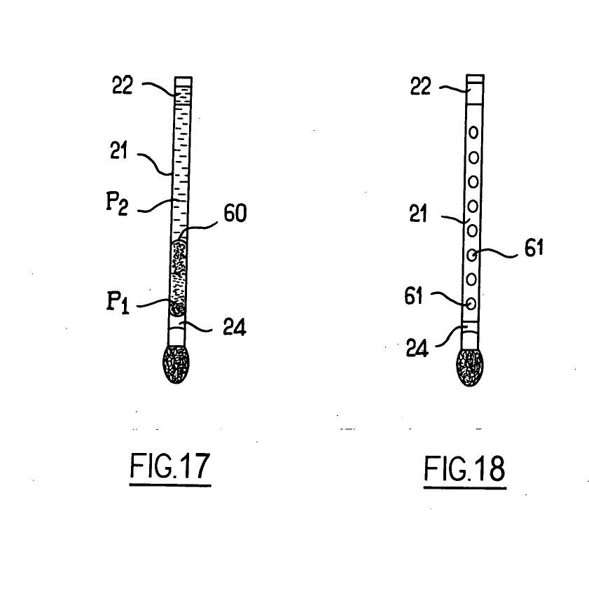

FIG. 17 shows a tube including two liquid reagents P₁ and P₂ that are present in the form of two phases each occupying a fraction of the length of the tube. The two reagents P₁ and P₂ are in contact with each other via an interface 60. One of the reagents may also be present in the form of at least one globule within the other phase, for example in the form of a plurality of globules 61 as shown in FIG. 18. This can make it possible, for example, to measure out the liquid using the tube as a pipette, or to improve the appearance of the applicator.

A plurality of different liquids can also be dispersed in the form of a plurality of globules in a single phase. The various globules can thus correspond to reagents of different colors and/or containing reagents at different concentrations and/or of different kinds.

When contained in the tube, it is also possible for the reagent P₂ to be solid, e.g. being constituted by a powder that is soluble in the liquid P₁, the substances P₁ and P₂ being separated from each other prior to use by a plug. The volume of the substance P₂ can be small enough to ensure that the substance P₂ can be dissolved easily in use. The number of substances present in the analyte-taking device can be greater than two, without thereby going beyond the scope of the present invention.

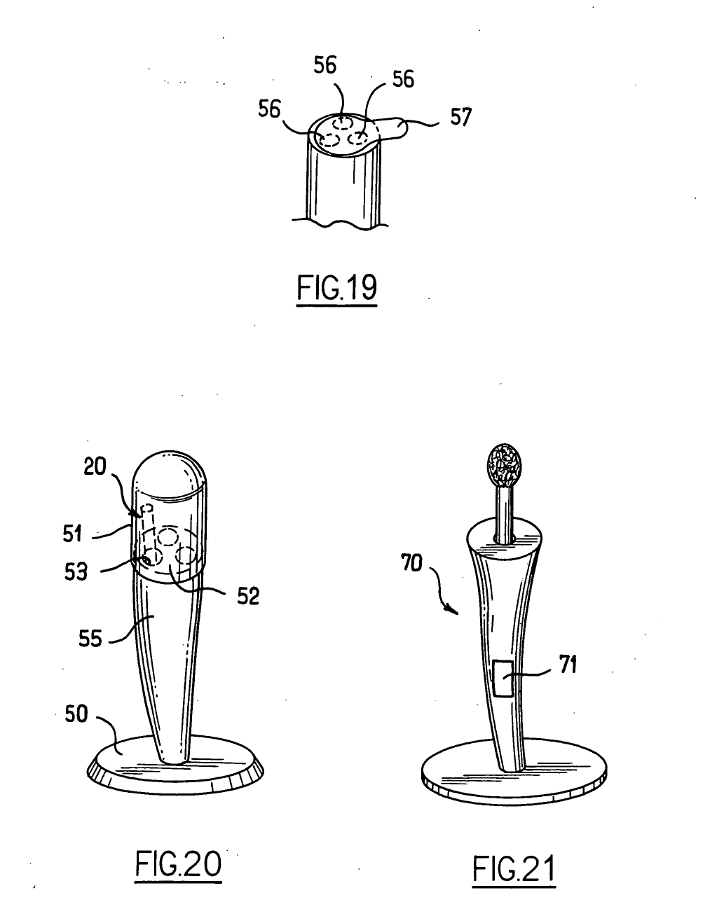

FIG. 19 shows the top end of a tube that contains three internal channels 56 by way of example, each containing a respective liquid and an associated plug. The tube is closeable at this end prior to use by a removable capsule 57 which can be stuck or heat-sealed on the tube, for example.

FIG. 20 shows a receptacle suitable for receiving an analyte-taking device. By way of example, such a receptacle can include a stand 50 supporting a body 55 whose top end is configured to enable a closure cap 51 to be fixed in place to close the receptacle in substantially leaktight manner. A support element 52 is disposed inside the body and having at least one orifice 53 enabling an analyte-taking device 20 to be engaged therein.

Thus, if so desired, after taking an analyte, the user may place the analyte-taking device in the receptacle. The presence of the cap 51 makes it possible to avoid the analyte-taking element drying out, for example.

It is also possible to use an analyte-taking device with a support 70 of the kind shown in FIG. 21, enabling the analyte-taking device to be held with its analyte-taking element visible. The support can include, for example, means 71 enabling the break-off end to be broken off when the analyte-taking device is put into place in the support 70. By way of example, these means 71 include a window giving access to the break-off end, or an element that is movable relative to the support and which applies lateral thrust on the break-off end when actuated. The tube need not have a break-off end but can merely have an end that is closed, with the support 70 being fitted with a blade or a spike, for example, serving to cut or pierce the tube so as to allow air to penetrate into the inside, and the liquid and the plug to be expelled when the analyte-taking device is used.

The invention is not limited to the embodiments described above. A device of the invention can have numerous applications. In addition to the applications described above, the invention makes it possible, amongst other things, to evaluate the activity of acid phosphatase, which can be representative of the extent to which the skin has been harmed by detergents, to evaluate catalase, or to evaluate barrier function deficiency or the buffering ability of the skin.

The end of the tube 21 remote from the end through which the substance leaves can, for example, be closed other than by a portion suitable for being broken off manually. For example, it can be closed by a plug or a piston.

The tube 21 can be made with an enlarged portion, e.g. in order to enable pressure to be exerted on the liquid so as to cause it to leave the tube.

The term “tube” is used to cover any body with preferably a generally elongate section that presents at least one internal channel capable of containing a liquid. The elongate section can optimally have a constant cross-section and a tube can present a longitudinal axis that is rectilinear or otherwise. The invention is not limited to a tube of circular outside section, nor is it limited to a tube made in accordance with the teaching of U.S. Pat. No. 5,702,035.

Throughout the description, including the claims, the terms such as comprising, including, having, or have should be understood as being synonymous with “comprising at least one” or “including at least one”, respectively, unless specified to the contrary.

Obviously, numerous modifications and variations of the present invention are possible in light of the above teachings. It is therefore to be understood that, within the scope of the appended claims, the invention may be practiced otherwise than as specifically described herein.

What is claimed as new and desired to be secured by Letters Patent of the United States is:

1. An analyte-taking device comprising:
a tube provided at one end with an analyte-taking element;
a plug inside the tube; and
at least one liquid contained in an inside space of the tube separated from the analyte-taking element at least by the plug, the plug being arranged, in use, to be expelled together with the liquid towards the analyte-taking element,
wherein the liquid is a reagent suitable for producing an observable reaction in the presence of an analyte picked up by the analyte-taking element.

2. The device according to claim 1, wherein the analyte-taking element is pre-impregnated with an analyte-taking liquid.

3. The device according to claim 1, wherein the analyte-taking liquid is selected from the group consisting of chloroform, ethyl acetate, alcohols, chlorine-containing solvents, acetone, short esters, aqueous solutions of methanol, and solutions of chloroform and ethanol.

4. The device according to claim 2, wherein the analyte-taking liquid is selected from the group consisting of chloroform, ethyl acetate, alcohols, chlorine-containing solvents, acetone, short esters, aqueous solutions of methanol, and solutions of chloroform and ethanol.

5. The device according to claim 4, wherein the analyte-taking element is pre-impregnated with an analyte-taking liquid.

6. The device according to claim 1, wherein the analyte-taking element is porous.

7. The device according to claim 1, wherein the analyte-taking element is fibrous.

8. The device according to claim 1, wherein the analyte-taking element is selected from the group consisting of a cotton bud, a foam bud, a flocked bud, a felt tip, and a tip made of ceramic or of sintered material.

9. The device according to claim 1, wherein the plug comprises a liquid selected from the group consisting of mineral oils, fluorine-containing substances, and silicones.

10. The device according to claim 1, wherein the plug comprises a powder selected from the group consisting of powders of microspheres of copolymers, of polyamide, of waxes, of silicas, and of silicones.

11. The device according to claim 1, wherein the inside space of the tube is defined, at an end remote from the plug by a portion that can be broken off, removed, perforated, or deformed.

12. The device according to claim 11, further comprising a retaining element for retaining the break-off portion on the analyte-taking device after it has been broken off.

13. The device according to claim 1, wherein the liquid in the tube has a volume in a range from 0.01 ml to 5 ml.

14. The device according to claim 1, wherein the liquid in the tube has a volume in a range from 0.05 ml to 1 ml.

15. The device of claim 1, wherein said plug is one of a liquid and a powder.

16. A kit for taking and analyzing an analyte, the kit comprising:
at least one analyte-taking device according to claim 1.

17. The kit according to claim 16, further comprising a box including at least one compartment in which said at least one analyte-taking device is housed.

18. The kit according to claim 16, further comprising at least one packaging bag containing said at least one analyte-taking device.

19. The kit of claim 16, wherein said plug is one of a liquid and a powder.

20. A method of detecting at least one of a presence and a concentration of at least one analyte at a surface of a tissue of an individual, the method comprising the steps of:
providing an analyte-taking device comprising a tube, a plug inside said tube, at least one analyte-taking liquid contained in an inside space of the tube defined at a first end by the plug, and an analyte-taking element at one end of the tube, separated from the analyte-taking liquid by the plug;
opening the tube so as to allow the analyte-taking liquid to leave the tube, the plug being suitable for being expelled together with the analyte-taking liquid;
taking at least one analyte with the analyte-taking element; and
putting the analyte into contact with a reagent suitable for producing an observable reaction in the presence of said analyte or in the presence of a determined concentration of the analyte.

21. A device for sampling an analyte, comprising:
a container having a first end which is open and a second end which is closed in a first position;
a removable plug inside said container, wherein said plug isolates a volume inside said container from said first open end in said first position;
a reagent inside said volume, said reagent being capable of reacting with said analyte; and
an element capable of receiving said analyte, said element being provided over said first open end of said container.

22. The device of claim 21, wherein said element comprises an absorbent material.

23. The device of claim 21, wherein said container is transparent.

24. The device of claim 21, wherein said container is a tube.

25. The device of claim 21, wherein said container includes a multilayer structure.

26. The device of claim 21, wherein said reagent produces a colored reaction with said analyte.

27. The device of claim 21, wherein said element is provided in an hermetically sealed packaging.

28. The device of claim 21, wherein said element is curved in shape, with a portion extending along a longitudinal axis which does not coincide with a longitudinal axis of the container.

29. The device of claim 21, wherein said reagent comprises at least two reagents in at least two different phases, each phase occupying a portion of said volume.

30. The device of claim 29, wherein said at least two reagents are liquid reagents.

31. The device of claim 29, wherein one of said at least two reagents is solid and another is liquid.

32. The device of claim 21, wherein said second end is movable from said first position to a second position which is open, said reagent being in communication with said element via said first end in said second position.

33. The device of claim 32, wherein said second end is capable of plugging said container after the second end has moved to said second position.

34. A system for sampling an analyte, comprising:
a packaging;
a plurality of analyte-taking devices provided in said packaging, each analyte-taking device comprising:
a tube containing a reagent capable of reacting with said analyte,
a first end which is open,
a second end which is closed in a first position, and
a removable plug inside said tube, each tube being coupled to an element comprising an absorbent material.

35. The system of claim 34, wherein said plug defines a closed volume for said reagent between said plug and said second end in said first position.

36. The device of claim 35, wherein said solution is in communication with said element via said first open end when the second end is in a second position.

37. The system of claim 36, wherein said second end is attached to said tube in said second position.

38. The system of claim 34, wherein said packaging comprises a box.

39. The system of claim 34, wherein said packaging comprises a string of bags, with an analyte-taking device in each of said bags.

40. The system of claim 34, wherein said packaging comprises:
a stand; and
a body mounted on said stand.

41. The system of claim 40, wherein each of said analyte-taking devices has a portion extending outside said body, and wherein said packaging further comprises a closure cap coupled to said body so as to cover said portions.
